(19) United States
(12) Patent Application Publication
(10) Pub. No.: US 2003/0211594 A1
(43) Pub. Date: Nov. 13, 2003

(54) Microalgae for remediation of waste and method of culturing the same

(76) Inventor: Donald Ian Rosebrook, Englewood, CO (US)

Correspondence Address: SHERIDAN ROSS PC, 1560 BROADWAY, SUITE 1200, DENVER, CO, 80202

(21) Appl. No.: 10/158,555

(22) Filed: May 29, 2002

Related U.S. Application Data
(60) Provisional application No. 60/378,754, filed on May 7, 2002.
(60) Provisional application No. 60/379,563, filed on May 10, 2002.

(51) Int. Cl. C12N 1/12

(52) U.S. Cl. 435/257.3

(57) ABSTRACT

A novel microalgal strain and progeny thereof, useful for the remediation of waste water, are disclosed. Also disclosed is a method of culturing these microalgae.

### CROSS-REFERENCE TO RELATED APPLICATIONS

This application claims the benefit of priority under 35 U.S.C. §119(e) from U.S. Provisional Application Serial No. 60/378,754 by Haerther et al., filed May 7, 2002, entitled “SYSTEM AND METHOD FOR REMEDIATION OF WASTE”, which is incorporated herein by reference in its entirety. This application also claims the benefit of priority under 35 U.S.C. §119(e) from U.S. Provisional Application Serial No. 60/379,563 by Rosebrook, filed May 10, 2002, entitled “MICROORGANISM FOR REMEDIATION OF WASTE AND METHOD OF CULTURING THE SAME”, which is incorporated herein by reference in its entirety.

### FIELD OF THE INVENTION

This invention generally relates to a strain of microalga that is particularly effective for use in the remediation of waste water, to adapted progeny strains thereof, and to methods of culturing these strains.

### BACKGROUND OF THE INVENTION

The last decade has witnessed a change in the production of livestock and dairy products from small, family owned units, to large, generally corporate owned farms. As a direct result of this evolution, large wastewater ponds have been constructed to consolidate waste handling and remediation. However, the increased production of these large farms has also resulted in increased waste which directly impacts air and water quality in the surrounding area.

Because of economic constraints, livestock production units typically utilize large anaerobic earthen or concrete storage basins. These large ponds can potentially be sources of air and water pollution. Anaerobic decomposition produces carbon dioxide; methane (one of the greenhouse gases); hydrogen sulfide (of concern because of its toxicity and odor); and ammonia. Odors from anaerobic processes are a threat to livestock produces in the U.S., and many other countries. The smell of nearby manure decomposition is unacceptable to a sufficiently large portion of our population and thus has created concerns throughout the livestock and dairy industry.

Aerobic treatment of waste can be achieved through use of a microbial agent whereby aerobic microbes use dissolved and suspended organic matter as a source of food. Microbial populations have been shown to be capable of either adsorbing, absorbing, or metabolizing a wide range of organic or inorganic compounds. These microbes produce oxygen as a byproduct of photosynthesis, along with other byproducts which may or may not be desirable in achieving waste remediation. As such, remediation of waste water using algal and/or bacterial cultures has been known in the art for many years. Nitrogen and carbon content can be reduced by cultivation of algae and bacteria in waste waters (Baumgarten et al., 1999, Appl. Microbiol. Biotechnol. 52:281-284) and growth of algae such as Chlorella species or Scenedesmus species in waste water reduces both chemical oxygen demand (COD) and biological oxygen demand (BOD) values below the discharge limits (Hammouda et al., 1995, Ecotoxicol. Environ Saf. 31:205-210). Microalgae are also known to remove various metals from waste waters (e.g., Chan et al., 1991, Biomed. Environ. Sci. 4:250-261). Bioremediation processes using algae typically include combining or culturing algae and/or bacteria with an aqueous waste material such as sewage, combined with additional steps such as mechanical aeration of the waste pond, injection of oxygen and/or carbon dioxide into the waste pond, management of the pH in the waste pond, multiple transfers to different holding ponds, steps of anaerobic pretreatment of the waste, and/or significant algae monitoring and growth regulation steps.

For example, U.S. Pat. No. 3,955,318 to Hulls, issued May 11, 1976 describes a process of purifying aqueous organic waste material by mixing algae with the waste under conditions whereby the mixture is aerated using a mixture of oxygen and carbon dioxide, combined with exposure to alternating, brief periods of light and darkness. In this system, the algae are supplied to the waste water from an outside source and can include any unicellular algae such as algae from Chlorophyta, Euglenophyta, Christophyta, Pyrrophyta, Cyanophyta and Rhodophyta.

U.S. Pat. No. 4,005,546 to Oswald, issued Feb. 1, 1977, describes a method of waste treatment wherein a body of aqueous waste containing algae is transferred through multiple ponds, with each pond being exposed to different conditions. In a preferred embodiment, the first pond containing waste and algae is open to light and air. The contents of the first pond are then transferred to a second pond that is also open to light and air, where additional algae nutrients are added and the pond is continuously agitated. Finally, the contents of the second pond are transferred to a pond that is shielded from light and dark. The algae in this system naturally occur in the waste water, although algae can be reintroduced from the third, dark pond back into the first pond.

U.S. Pat. No. 4,209,388 to DeFraites, issued Jun. 24, 1980, describes a method of waste treatment which includes a first process of introducing waste into an algae containing pond which is either aerated, facultative, or a combined aerobic and anaerobic pond, followed by transfer of the waste water to a second pond where the algae are deprived of nutrients and sunlight, causing algal death and settling. The waste water is then transferred to a third pond to separate the water from the dead algae. The source and types of algae used in this system are not disclosed.

U.S. Pat. No. 4,267,038 to Thompson, issued May 12, 1981, describes a purification system for waste water in which includes steps of removal of solids from the waste water as sludge, digestion of the sludge and recombining with the waste water, a step of anaerobic, bacterial oxidation of waste water organics, followed by nutrient stabilization, nitrification, denitrification and reaeration, and then transfer of the water from the anaerobic tanks to one or more tanks containing algae and aerobic bacteria. The treated water can be channeled through a variety of tank combinations, including recycling back through anaerobic or aerobic tanks, cycling through series of aerobic tanks, and dewatering of algae for collection of the algae as a useable endproduct. The algae containing tanks are controlled with brief cycles of artificial light and the introduction of turbulence into the tanks.

U.S. Pat. No. 4,432,869 to Groeneweg et al., issued Feb. 21, 1984, describes a process for the treatment of liquid agricultural wastes by using an algae/bacteria mixture, followed by exposure of the waste to a rotifer culture. The step of culturing the algae and bacteria mixture includes controlling the pH of the culture to inhibit multiplication of rotifers during the first stage, as well as aeration of the treatment pond. The pH is then altered to allow only for the culture of rotifers in the water. The waste water is also anaerobically pretreated prior to introduction to the algae/bacteria mixture. The algae that can be used in this system include species of Chlorella r Scendesmus.

U.S. Pat. No. 4,966,713 to Keys et al., issued Oct. 30, 1990, describes a process for treating waste water from a food processing plant using a flocculant comprising a crude algal composition or processed algae and an acidic pH. The process produces a floc which is then separated from the water. The algae source can include Rhodophyceae, Cyanophyceae, Cholorophyceae and Phaeophyceae.

U.S. Pat. No. 6,350,350 to Jensen et al., issued Feb. 26, 2002, describes a process for removing pollutants from waste water by running the waste water over a bed of algae in an attached periphyton bed. The algae are then harvested for use in a mix with a shredded paper product to produce a pulp.

In addition to these processes, various algal species have been described as being useful for bioremediation methods, being capable of utilizing waste products, or as naturally occurring within waste waters. Such algae include Chlorella species and Scenedesmus species (see, e.g., Matusiak et al., 1977, Acta Microbiol. 26:79-93; Chrost et al., 1975, Acta Microbiol. Pol B 7:231-236; Matusiak, 1976, Acta Microbiol Pol 25:233-242; Chan et al., 1991, supra; Baumgarten et al., 1999, supra; Hammouda et al., 1995, supra). U.S. Pat. No. 3,882,635 to Yamanaka et al., issued May 13, 1975, describes Prototheca sphaerica FERM P-1943 as being capable of growing on a wide variety of waste waters of the food industry. This species is alleged to be superior to Chlorella species with regard to the carbon sources on which these algae can grow.

However, there are a number of disadvantages in current aerobic treatment methods. One current disadvantage is that many of the microbes presently utilized in waste treatment methods are particularly sensitive to heat and light conditions, and such microbes only flourish in optimum light and temperature conditions. In cold weather climates where there are greater variations in daily temperature highs and lows, many microbes do not flourish, particularly in the colder winter months. Accordingly, the rate at which waste is remediated greatly drops off during the winter months. Another factor which presently limits most aerobic treatment processes is that there must be certain existing levels of dissolved oxygen and water available in order for the microorganisms to be metabolically active. In agricultural operations, the waste ponds often have a thick hard and dry upper crust which carries little or no oxygen and water. As a result, aerobic treatment for waste under these conditions is very inefficient. Another factor which limits the effectiveness of current aerobic processes is that many microbe species are unable to effectively adapt to new environments, and the wastes which the microbes encounter may not be optimum for sufficient growth of the microbes.

Therefore, there remains a need in the art for simplified and improved methods of bioremediation that take advantage of the aerobic processes for waste treatment.

### SUMMARY OF THE INVENTION

One embodiment of the present invention relates to an isolated microalga selected from the group consisting of: (a) Chlorella sp., strain rosebrokii AgSmart 100 (AG-SMART 100 ATCC No. ______); (b) Chlorella sp., strain rosebrokii AgSmart 200 (AG-SMART 200 ATCC No. ______); and (c) a microalga derived from any of the microalgae of (a) or (b).

Another embodiment of the present invention relates to a fermentation culture, comprising culture medium and microalgae of a strain selected from the group consisting of: Chlorella sp., strain rosebrokii AgSmart 100 (AG-SMART 100 ATCC No. ______), Chlorella sp., strain rosebrokii AgSmart 200 (AG-SMART 200 ATCC No. ______), and microalgae derived from either of the strains. In one aspect, the culture medium has a dissolved oxygen content of at least about 5 mg dissolved oxygen per liter culture medium. In another aspect, the culture medium has a dissolved oxygen content of at least about 10 mg dissolved oxygen per liter culture medium. In yet another aspect, the culture medium has a dissolved oxygen content of at least about 25 mg dissolved oxygen per liter culture medium. In one aspect, the culture medium comprises fermented animal manure and water.

Yet another embodiment of the present invention relates to a process for culturing microalgal cells for use in a remediation process. The process comprises culturing microalgal cells at a temperature of from about 20° C. to about 33° C. in a culture medium at a pH of from about pH 7.5 to about pH 8.4 in a culture medium comprising water and a primary growth medium stock, wherein the primary growth medium stock comprises fermented animal manure and water. In one aspect, the primary growth stock is produced by a method comprising: (a) mixing animal manure with water at a ratio of from about 1-5 parts manure to about 200 part water to form a manure mixture; (b) fermenting the manure mixture at a temperature of from about 20° C. to about 24° C., for at least about three days to form a concentrate; and (c) diluting the concentrate with water to form the primary growth medium having a turbidity reading of at least about 20 ntu. In another aspect, the culture medium further comprises a source of finely chopped meat. In one aspect, the water in the culture medium comprises water from a source of waste water. In one aspect, the microalgal cells are cultured at a temperature from about 24° C. to about 26° C. In another aspect, the microalgal cells are cultured until the culture medium has a dissolved oxygen content of from about 5 mg dissolved oxygen per liter of culture medium to about 25 mg dissolved oxygen per liter of culture medium. In another aspect, the microalgal cells are from the genus Chlorella. In a preferred aspect, the microalgal cells are selected from the group consisting of: Chorella sp., strain rosebrokii AgSmart 100(AG-SMART 100 ATCC No. ______), Chlorella sp., strain rosebrokii AgSmart 200 (AG-SMART 200 ATCC No. ______),and microalgae derived from any of the strains.

### DETAILED DESCRIPTION OF THE INVENTION

The present invention generally relates to a novel microalga and to progeny derived from this microalga, all of which have been found to be particularly effective for use in a system and method for the remediation of waste water (e.g., water containing animal and/or human waste products). The microalga is believed to be of the genus Chlorella and has been deposited with the American Type Culture Collection, as discussed in detail below. A method and system for remediation of waste water using the microalgae of the present invention are described in Provisional Application Serial No. 60/378,754, filed May 7, 2002, by Haerther et al., entitled “System and Method for Remediation of Waste” (incorporated herein by reference in its entirety), and in copending U.S. application Ser. No. ______, also by Haerther et al. and also entitled “System and Method for Remediation of Waste”, filed on May 29, 2002 (incorporated herein by reference in its entirety).

Accordingly, one embodiment of the present invention relates to an isolated microalga having the identifying characteristics of any of the specific microalgal strains described herein and deposited with the American Type Culture Collection, and any progeny derived from such microalgal strains. The microalgae of the present invention have been shown to be particularly useful for waste remediation processes. The microalgae of the present invention are believed to be new microalgal strains, and possibly represent a new species of algae of the genus Chlorella, and are generally referred to herein as Chlorella sp., strain rosebrokii. If it is later determined that this these microalgal strains are members of a new species of Chlorella, then the species will be named Chlorella rosebrookii. Two different substrains of the microalga of the present invention have been deposited with the American Type Culture Collection (ATCC), Manassas, Va. 20108 USA, under the terms of the Budapest Treaty on the International Recognition of the Deposit of Microorganisms for the Purpose of Patent Procedure, on ______, 2002 (received by the ATCC on May 22, 2002). The parent strain, denoted herein as Chlorella sp., strain rosebrokii AgSmart 100 (deposited as Chlorella rosebrookii AG-SMART 100), has received ATCC No. ______. One adapted progeny strain of the parent strain, denoted herein as Chlorella sp., strain rosebrokii AgSmart 200 (deposited with the ATCC as Chlorella rosebrookii AG-SMART 200; previously denoted as Chlorella sp., strain rosebrokii AgSmart 100.1 in U.S. Provisional Application Serial No. 60/379,563, entitled “Microorganism for Remediation of Waste and Method of Culturing the Same” and filed May 10, 2002), has received ATCC No. ______.

Chlorella sp., strain rosebrokii AgSmart 100(ATCC No. ______) is characterized as a unicellular green algae which has the morphology of the genus Chlorella, and which is of a currently unknown species. The cells are spherical and approximately 0.4μ in size, when cultured on normal fermentation medium made according to normal production parameters (e.g., using fermented animal waste in a primary growth medium (described below)). The cells contain a single chloroplast and one pyrenoid. Reproduction occurs via autospores. The cells were originally isolated from a bog 20 miles north of Liberal, Kans., in the United States of America. The microalgal cells of the present invention were growing in a small pond which was fed by water from a small spring. The cells were growing in and around water plants. The present inventor sampled the water and isolated the microalga of the present invention (parent strain Chlorella sp., strain rosebrokii AgSmart 100) from the water sample.

Using Modern Biology, Moon et al., 1960, the isolated microalga was classified as belonging to the class of algae known as Chlorophyceae, which are green, fresh water algae. The present inventor then further classified the isolated microalga as belonging to the genus Chlorella, based on morphological analysis and comparison to the literature. A sample of Chlorella vulgaris was obtained from the American Type Culture Collection (ATCC) which was compared to the isolated Chlorella sp., strain rosebrokii AgSmart 100 under the same growth conditions. As compared to C. vulgaris, Chlorella sp., strain rosebrokii AgSmart 100 had cells of a similar morphology, except that Chlorella sp., strain rosebrokii AgSmart 100 cells were slightly different in the construction of the chloroplast and nucleus, which were generally smaller than the same organelles of C. vulgaris when the two strains were grown on the same fermentation medium. The characteristics of the microalgal strain of the present invention were also compared to published characteristics of three species of Prototheca (P. sphaerica, P. stagnora, P. wickerhamii). As compared to the Prototheca species, the microalga of the present invention is smaller in diameter by approximately 2 microns.

Utilizing the standard fermentation medium described below (produced from fermented animal waste), the Chlorella sp., strain rosebrokii AgSmart 100 was compared to Chlorella vulgaris described above with regard to growth characteristics. The production of oxygen by C. vulgaris under natural sunlight was on average about 3 mg/L (3 mg dissolved oxygen per liter medium), whereas the oxygen production by Chlorella sp., strain rosebrokii AgSmart 100 under the same conditions averaged 11 mg/L. When compared to C. vulgaris cultured in the absence of light, Chlorella sp., strain rosebrokii AgSmart 100 produced an average of 5 mg/L of oxygen whereas the C. vulgaris strain did not produce any measurable oxygen. In fact, the oxygen in the standard fermentation medium was depleted by C. vulgaris when cultured in the dark, dropping from 5 mg/L to 1 mg/L of dissolved oxygen 6 hours after being deprived of sunlight.

Moreover, the Chlorella sp., strain rosebrokii AgSmart 100 strain initiated growth at about 36° F., whereas the C. vulgaris strain required a temperature of at least 55° F. to initiate growth. Growth was measured in these experiments by measuring oxygen production and density of chlorophyll. When the C. vulgaris was grown in the standard fermentation medium as described below, the pH stabilized at 9.8 after 72 hours of growth, whereas the pH of the fermentation medium containing the Chlorella sp., strain rosebrokii AgSmart 100 strain stabilized at pH 8.4 after 72 hours of growth.

Chlorella sp., strain rosebrokii AgSmart 100 and its progeny grow by respiration, utilizing glucose as a carbon source, for example, and produce six units of oxygen, six units of carbon dioxide, and 12 units of water for each molecule of glucose utilized.

The genomic DNA of Chlorella sp., strain rosebrokii AgSmart 100 has been analyzed on a polyacrylamide gel and produces bands at 1778 kb, 1514 kb, 1148 kb, 933 kb, 708 kb, 575 kb and 380 kb.

The progeny strain described herein, denoted herein as Chlorella sp., strain rosebrokii AgSmart 200 (AG-SMART 200, ATCC No. ______) is exemplary of progeny strains which have been derived from the parent strain (Chlorella sp., strain rosebrokii AgSmart 100), but which have been grown in specific waste treatment conditions (i.e., waste from particular animal sources and particular waste treatment facilities) such that these strains have adapted to the specific waste conditions and now differ in their growth characteristics and requirements as compared to the parent strain and to each other. For example, Chlorella sp., strain rosebrokii AgSmart 200 was derived from the parent strain through an extended period of growth of the parent strain (i.e., greater than 12 months) on waste water from a specific site containing porcine waste which is being treated using the novel remediation system described in U.S. Provisional Application Serial No. 60/378,754, filed May 7, 2002, by Haerther et al., supra and in U.S. application Ser. No. ______, filed on May 29, 2002, by Haerther et al., supra. Chlorella sp., strain rosebrokii AgSmart 200 (AG-SMART 200) represents one adapted substrain of the parent strain that is now believed to be especially adapted for growth on the particular waste source and site on which it was selected (e.g., it grows exceptionally well, thriving, under those conditions). Therefore, encompassed by the present invention are any progeny of the parent strain Chlorella sp., strain rosebrokii AgSmart 100 or Chlorella sp., strain rosebrokii AgSmart 200, which can be derived by extended culture and adaptation to any specific waste water source and/or waster water treatment facility. In addition, classical mutagenesis methods for deriving altered strains of microorganisms from a parent strain are known in the art.

The primary growth medium used in the process of the present invention can be any medium suitable for culturing microalgae, and particularly for culturing Chlorophyta algae, and more particularly for culturing microalgae of the genus, Chlorella, and even more particularly, for culturing the novel microalgae disclosed herein (e.g., AG-SMART 100 (ATCC No. ______) or AG-SMART 200 (ATCC No. ______) and progeny thereof). According to the present invention, a suitable primary growth medium generally comprises a source of assimilable organic carbon, a source of assimilable nitrogen and appropriate salts and trace metals. In one embodiment, the medium is suitable for growing and maintaining a substantially pure culture of the microalgae of the present invention (e.g., substantially free of contaminating microorganisms and/or impurities that might negatively impact the growth of the microorganisms). In another embodiment, the medium is suitable for growing and maintaining the microalgae for use in a remediation process, wherein growth of other microorganisms (e.g., bacteria useful in remediation) can occur.

Sources of assimilable carbon which can be used in a suitable primary growth medium include, but are not limited to, sugars and their polymers, including, dextrin, sucrose, maltose, lactose, glucose, fructose, mannose, sorbose, arabinose and xylose; fatty acids; organic acids such as acetate; primary alcohols such as ethanol and n-propanol; and polyalcohols such as glycerine. The concentration of a carbon source, such as glucose, in the fermentation medium should promote cell growth, but not be so high as to repress growth of the microalgae.

Sources of assimilable nitrogen which can be used in a suitable primary growth medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids.

The effective primary growth medium can contain other compounds such as inorganic salts, vitamins, trace metals or growth promoters. Such other compounds can also be present in carbon, nitrogen or mineral sources in the effective medium or can be added specifically to the medium. The fermentation medium can also contain a suitable phosphate source, including both inorganic and organic phosphate sources.

A preferred primary growth medium useful for culturing microalgae of the present invention is prepared from animal wastes (e.g., animal manure). This primary growth medium is particularly useful when the microalgae of the present invention are used in a waste remediation process, although use of this primary growth medium is not limited to waste remediation processes. Moreover, other suitable growth mediums, including any art recognized or known medium for culturing a microalgae such as Chlorella, can be used to culture the microalgae of the present invention. In this embodiment, when animal waste is used to prepare the medium, preferably, the animal manure used in the primary growth medium is from domestic livestock, such as from cattle. In a more preferred embodiment, the manure is collected from cattle which are fed a diet that is high in carbohydrates and protein and that is supplemented with minerals. For example, a preferred diet to feed cattle from which the manure is collected includes alfalfa, corn gluten or corn, a protein source (e.g., Rumensm), calcium, sodium, potassium, urea and a sugar source, such as molasses. Such diets are typically fed to cattle on feed lots, for example. The present inventors have found that manure collected from such animals results in an excellent source of material for the primary growth medium for the microalgae of the present invention.

Primary Growth Medium Stock (Added to the Growth Tanks in which the Microalgae Are Cultured) Suitable for Culture for Remediation Processes:

To prepare the primary growth medium stock (also referred to herein as the primary growth medium), a concentrate is first prepared. Briefly, animal manure as described above is mixed with water at a ratio of about 1-5 parts manure to about 200 parts water, and preferably about 1-4 parts manure, and more preferably about 1-3 parts manure, and more preferably about 1-2 parts manure, and more preferably about 1 part manure to about 200 parts water. The water can be supplied from any source, including, but not limited to, well water, tap water, purified water, and deionized water, and is preferably low in chlorine. The water and manure mixture are fermented at 70-75° F. (˜20-24° C.), with constant stirring, under covered conditions. The mixture is fermented for at least about three days (optimum length of time), but can be fermented for longer, such as for four, five, or up to about six days, maximally, to form a concentrate for the primary growth medium stock. If the temperature is raised, such as to 100-110° F., the mixture can be fermented in about two days, and maximally up to about three days. The concentrate is typically fermented until a turbidity reading of from about 75 ntu (nephlometric turbidity units) to about 80 ntu is reached, using an HACH Company turbidity meter set at the 0 to 200 ntu range.

The primary growth medium stock is added to growth tanks containing the microalgae at an average turbidity of from about 20 ntu to 25 ntu, measured as described above. This turbidity is achieved by diluting the concentrate described above with water to the appropriate level prior to introduction of the medium to the growth tanks. The primary growth medium stock can be more or less dilute than this average turbidity reading, depending on length of time the growth tank has been established, the source of water or other supplemental food materials that may be added to the growth tank, and the general health of the microalgae culture in the growth tank. Alternatively, the amount of nutrients added to the growth tanks can be adjusted by modifying the amount of primary growth medium stock that is added to the growth tanks. The primary growth medium stock can be stored in a tank or other container near the microalgae growth tanks for ease of use. Typically, the primary growth medium stock is stirred thoroughly either continuously or at least just prior to delivery to the growth tanks, since the medium contains particulate matter. The primary growth medium stock can also be filtered (e.g., using a 50 mesh screen) prior to introduction to the growth tank to remove large particulate matter. The primary growth medium stock produced by this method contains sufficient protein and carbohydrate to sustain the critical growth levels of the microalgae of the present invention during a remediation process, when supplied as described below.

Microalgae Growth Tanks:

The microalgae growth tanks (e.g., fermentation or culture tanks) contain the microalgae to be cultured (e.g., microalgae of the present invention); the primary growth medium stock diluted in a water source to provide the culture medium (fermentation medium); and may also contain additional sources of supplemental nutrients (described below).

The primary growth medium stock is typically added to the fermentation tanks (i.e., the microalgae growth tanks) at a concentration of from about 2 to about 15 gallons primary growth medium stock per 1000 gallons total volume culture medium in the growth tank, and more preferably, from about 2 to about 10 gallons primary growth medium stock per 1000 gallon growth tank, and more preferably, from about 5 to about 10 gallons primary growth medium stock per 1000 gallon growth tank, and even more preferably, from about 5 to about 8 gallons primary growth medium stock per 1000 gallon growth tank.

In one embodiment of the invention, in addition to adding the primary growth medium stock to the growth tanks, a supplemental food source can be added to enhance the growth of the microalgae in the tanks. A particularly preferred supplemental food source includes a finely chopped, fresh source of meat and blood (skin, claws, feathers, hair scales and/or bones are preferably removed). Alternatively, any supplemental source of protein and other nutrients can be used. This supplemental food source is added to the fermentation medium in the growth tanks at a rate of about one quarter of a gallon to about 1 gallon per 1000 gallon total culture medium in the growth tank per day, or as needed to achieve the necessary microalgae growth rates.

In one embodiment, in addition to being fed with the primary growth medium stock described above (and the supplemental food source, if used), the fermentation medium in the growth tanks can be further supplemented with waste water from the waste water source being treated, which provides additional nutrients to the growth tank to support the microalgae. In this embodiment, water from the waste water source (e.g., lagoon or water treatment tank) is pumped from the source into the microalgae growth tanks. Typically, the waste water is added to the growth tanks at a rate of about 2 gallons to about 5 gallons per 1000 gallon growth tank per day. This water, being supplied from the waste water source, contains additional nutrients, including protein and carbon, which supplement the growth of the microalgae. In one embodiment, if the waste water being treated contains sufficient nutrients to sustain the microalgal growth, it may be possible to use less of the primary growth medium stock to feed the growth tanks in the earlier stage of the remediation process, and the supplemental food source described above may be omitted altogether. As the waste water becomes cleaner as a result of the microalgal treatment, the amount of primary growth medium stock can be increased in the growth tanks and supplemental food source can be added, as necessary to maintain the microalgal growth. It is believed that culturing the microalgae in a source of the waste water into which the microalgae will eventually be added for remediation enables the microalgae to adapt to the waste water conditions (e.g., the environment in which the remediation will take place) in advance and will result in a more robust remediation process.

Over time, as the Chlorella sp., strain rosebrokii AgSmart 100 (parent strain AG-SMART 100, ATCC No. ______) grows in fermentation medium that is fed, at least in part, using waste water from the waster water source to be treated, the present inventor has found that the strain will adapt to the waste water source and develop into substrains that are particularly well suited for growth on that particular waste water source. As such, the microalgae of the present invention can be effectively subcultured to produce, for example, one substrain that grows especially well on bovine waste and another substrain that grows especially well on human waste. Further, substrains that grow particularly well on one specific site versus another (e.g., one porcine waste source versus a different porcine waste source) can be developed. Effectively, the culture method of the present invention allows for the derivation of specialized substrains of the Chlorella sp., strain rosebrokii AgSmart 100 which provide individual remediation sites with a highly effective bioremediation system. By way of example, as discussed above, Chlorella sp., strain rosebrokii AgSmart 200 (AG-SMART 200, ATCC No. ______) was derived from the parent strain by extended culture on porcine waste.

The water used to make up the volume of the fermentation medium in the growth tank to which the primary growth medium stock, supplemental food source, and/or waste water from the remediation source are added can be from any source, including, but not limited to, well water, tap water, purified water, and deionized water. This water should be free from agents that are toxic or inhibitory to the growth of the microalgae, and preferably, is low in chlorine.

In one embodiment of the invention, the growth tanks (fermentation tanks), where the microalgae of the present invention are cultured, are used to regularly (e.g., daily) supply the algae and oxygenated water from the growth tank to a waste water source (e.g., a lagoon or other waste depository). In this embodiment, the primary growth medium stock is typically added to the fermentation tanks (i.e., the microalgae growth tanks) at a concentration of from about 3 to about 15 gallons primary growth medium stock per 1000 gallon total culture medium in the growth tank per day, and more preferably, from about 5 to about 10 gallons primary growth medium stock per 1000 gallon growth tank per day, and even more preferably, from about 5 to about 8 gallons primary growth medium stock per 1000 gallon growth tank per day. The primary growth medium stock is introduced into a source of water in the growth tanks, as described above, in which a microalgae culture of the present invention can already be established, or to which microalgae of the present invention are to be added (described below). The contents of the growth tanks (e.g., microalgae, oxygenated growth medium and other components present in the growth tanks) are emptied into the waste water to be treated at a rate of from about 500 to about 750 gallons per day. Therefore, in this embodiment, the growth tanks are maintained at the desired volume suitable for culture of the microalgae and production of oxygen by the microalgae, with added water and the primary growth medium stock on a daily basis as described above. Additional nutrients can be added to the growth tanks as needed to maintain the microalgae.

The temperature of the medium in the growth tanks is preferably maintained at from about 70°-90° F. (˜20°-33° C.), and typically, the temperature is maintained at a range of from about 75°-78° F. (˜24°-26° C.).

The pH of the growth tank is maintained at between about pH 7.5 to about pH 8.4 for optimum growth and health of the microalgae. It is preferable to maintain the culture within this pH range and monitor the tank to achieve a dissolved oxygen concentration of from about 5 mg dissolved oxygen per liter medium (˜5 mg/L) to about 25 mg/L (e.g., 5 mg/L, 10 mg/L, 15 mg/L, 20 mg/L, 25 mg/L and whole integers in mg/L between these points). The growth tank can be allowed to exceed 25 mg/L dissolved oxygen, but monitoring of the tank should continue to ensure the health of the biological system. If the growth tank drops below or above this level of dissolved oxygen, the conditions in the tank are corrected, such as by adding more or less primary growth medium stock to boost or reduce the algal growth, respectively; or by emptying more or less of the tank volume (or emptying more or less often) into the waste water source to reduce or increase the total algal concentration in the growth tank.

The culture medium in the growth tank (e.g., primary growth medium stock, water source, any supplemental nutrients and microalgal culture) is typically not aerated or stirred. The present inventor has found that the microalgae of the present invention grow well under these conditions. Therefore, in a preferred embodiment, the growth tanks are not stirred or otherwise aerated. In another embodiment, the growth tanks can be aerated.

The culture medium is exposed to a source of light for between about 16 and about 22 hours per day, with between 2 and 8 hours of darkness every 24 hours, and more preferably between 2 and 6 hours (18-22 hours of light), and more preferably between 4 and 6 hours (18-20 hours of light), and most preferably about 4 hours of darkness (about 20 hours of light) every 24 hours. In one embodiment, the culture is supplied with natural sunlight, such as in a green house, and supplemented with artificial light as needed to make up the required amount of light every 24 hours. The source of artificial light can be any suitable source, and is preferably a source which provides light that mimics natural sunlight. In one embodiment, a light source providing blue spectrum ultraviolet light is used.

The microalgae of the present invention are initially added to the growth tanks (˜1000 gallons total culture medium) as an inoculum of from about 5 to about 200 gallons of a “starter culture”. The starter culture is prepared by growing microalgae of the present invention in a smaller volume of fermentation medium that is substantially similar to that used in the large growth tanks, wherein the microalgae reach a density measured as turbidity in the medium of from about 5 ntu to about 20 ntu before being added to the large growth tanks (or that is any suitable microalgae culture medium). Preferably, the microalgal culture is grown to a turbidity of about 20 ntu or greater, at a pH range of from about 7.5 to about 8.4, with the higher pH range being preferred to establish the microalgae in the growth tank. Typically, a period of about three days is required to establish a sufficient concentration of microalgae in the large, 1000 gallon growth tanks under controlled environmental conditions.

Once the culture is established in the growth tanks, it is preferable to maintain the culture at a density sufficient to maintain the dissolved oxygen concentration in the fermentation medium at a level of from about 5-10 mg dissolved oxygen per liter medium (˜5-10 mg/L) to about 25 mg/L, at a pH of between about pH 7.5 to about pH 8.4. Preferably, the microalgae are cultured at a density sufficient to maintain the dissolved oxygen content at up to about 25 mg/L and at a pH of up to about 8.4. The dissolved oxygen content may be allowed to exceed 25 mg/L, but the pH generally should not be allowed to exceed pH 8.4. The density, dissolved oxygen and pH of the culture medium can be regulated by several factors including, but not limited to, the rate of turn-over of the growth tank culture into the waste water source, and the rate of addition and/or concentration of primary growth medium, supplemental food source and/or waste water added to the growth tanks.

Waste Water Treatment:

In one embodiment of the present invention, the microalgae of the present invention are used in a method of waste remediation. In this embodiment, cultured microalgae from the growth tanks as described above are introduced into a source of waste water to be treated (e.g., a waste lagoon, a water treatment storage tank) in an amount sufficient to effect remediation of the waste in the water. For example, in one embodiment, a waste water source is monitored to include an amount of microalgae of the present invention sufficient to achieve a level of dissolved oxygen in the waste water source of between about 2 mg/L and about 10 mg/L, at a pH of between about pH 7.5 and pH 8.4. In one embodiment, a standard lagoon containing waste water is treated with from about 500-1000 gallons per day of culture medium containing algae from a growth tank as described above. Details of an exemplary remediation process in which the microalgae of the present invention are particularly useful are described in copending U.S. patent application Ser. No. ______, by Haerther et al., filed on May 29, 2002, supra (incorporated herein by reference in its entirety).

In another embodiment of the present invention, the microalgae of the present invention are cultured in a medium comprising a source of assimilable organic carbon, a source of assimilable nitrogen and appropriate salts and trace metals, at a pH of from about pH 7.0 to about pH 8.5, at a temperature of from about 20° C. to about 37° C., with or without additional aeration to the culture medium.

The microalgae of the present invention can be used for any suitable purpose other than bioremediation, including as a research tool and to produce useful compounds (e.g., vitamins, bioactive molecules). Microalgae of the present invention can also be genetically modified. Genetic modification of a microalga can be accomplished using classical strain development and/or molecular genetic techniques (e.g., recombinant technology). Such techniques are generally disclosed, for example, in Sambrook et al., 1989, Molecular Cloning: A Laboratory Manual, Cold Spring Harbor Labs Press. The reference Sambrook et al., ibid., is incorporated by reference herein in its entirety. A genetically modified microalga can include a microalga in which nucleic acid molecules have been inserted, deleted or modified (i.e., mutated; e.g., by insertion, deletion, substitution, and/or inversion of nucleotides), in such a manner that such modifications provide the desired effect within the microalga.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated microalga selected from the group consisting of:
a. Chlorella sp., strain rosebrokii AgSmart 100 (AG-SMART 100 ATCC No. ______);
b. Chlorella sp., strain rosebrokii AgSmart 200 (AG-SMART 200 ATCC No. ______);and
c. a microalga derived from any of said microalgae of (a) or (b).

2. The isolated microalga of claim 1, wherein said microalga is Chlorella sp., strain rosebrokii AgSmart 100 (AG-SMART 100 ATCC No. ______).

3. The isolated microalga of claim 1, wherein said microalga is Chlorella sp., strain rosebrokii AgSmart 200 (AG-SMART 200 ATCC No. ______).

4. A fermentation culture, comprising culture medium and microalgae of a strain selected from the group consisting of: Chlorella sp., strain rosebrokii AgSmart 100 (AG-SMART 100 ATCC No. ______), Chlorella sp., rosebrokii AgSmart 200 (AG-SMART 200 ATCC No. ______), and microalgae derived from either of said strains.

5. The fermentation culture claim of 4, wherein said culture medium has a dissolved oxygen content of at least about 5 mg dissolved oxygen per liter culture medium.

6. The fermentation culture of claim 4, wherein said culture medium has a dissolved oxygen content of at least about 10 mg dissolved oxygen per liter culture medium.

7. The fermentation culture of claim 4, wherein said culture medium has a dissolved oxygen content of at least about 25 mg dissolved oxygen per liter culture medium.

8. The fermentation culture of claim 4, wherein said culture medium comprises fermented animal manure and water.

9. A process for culturing microalgal cells for use in a remediation process, comprising culturing microalgal cells at a temperature of from about 20° C. to about 33° C. in a culture medium at a pH of from about pH 7.5 to about pH 8.4 in a culture medium comprising water and a primary growth medium stock, wherein said primary growth medium stock comprises fermented animal manure and water.

10. The process of claim 9, wherein said primary growth stock is produced by a method comprising:
a. mixing animal manure with water at a ratio of from about 1-5 parts manure to about 200 part water to form a manure mixture;
b. fermenting said manure mixture at a temperature of from about 20° C. to about 24° C., for at least about three days to form a concentrate; and
c. diluting said concentrate with water to form said primary growth medium having a turbidity reading of at least about 20 ntu.

11. The process of claim 9, wherein said culture medium further comprises a source of finely chopped meat.

12. The process of claim 9, wherein said water in said culture medium comprises water from a source of waste water.

13. The process of claim 9, wherein said microalgal cells are cultured at a temperature from about 24° C. to about 26° C.

14. The process of claim 9, wherein said microalgal cells are cultured until said culture medium has a dissolved oxygen content of from about 5 mg dissolved oxygen per liter of culture medium to about 25 mg dissolved oxygen per liter of culture medium.

15. The process of claim 9, wherein said microalgal cells are from the genus Chlorella.

16. The process of claim 9, wherein said microalgal cells are selected from the group consisting of: Chlorella sp., strain rosebrokii AgSmart 100 (AG-SMART 100 ATCC No. ______), Chlorella sp., strain rosebrokii AgSmart 200 (AG-SMART 200 ATCC No. ______), and microalgae derived from any of said strains.

17. The process of claim 9, wherein said microalgal cells are Chlorella sp., strain rosebrokii AgSmart 100 (AG-SMART 100 ATCC No. ______).

18. The process of claim 9, wherein said microalgal cells are Chlorella sp., strain rosebrokii AgSmart 200 (AG-SMART 200 ATCC No. ______).
